(19) United States
(12) Patent Application Publication
(10) Pub. No.: US 2009/0056716 A1
(43) Pub. Date: Mar. 5, 2009

(54) Cool air inhaler and methods of treatment using same

(75) Inventor: Frank J. Carrier, Shrewsbury, MA (US)

Correspondence Address: Nields & Lemack, 176 E. Main Street, Suite #5, Westboro, MA, 01581, US

(73) Assignee: Atlantic Research Group LLC

(21) Appl. No.: 11/899,110

(22) Filed: Sep. 4, 2007

(51) Int. Cl. A61M 16/10; A61M 15/00

(52) U.S. Cl. 128/204.15

(57) ABSTRACT

An inhaler device and a method of treating various symptoms associated with the airway and/or throat and/or respiratory system of a patient. The device is particularly designed to reduce swelling and inflammation of the larynx and or upper respiratory track, such as swelling and/or inflammation that result from croup, laryngitis's, laryngotracheobronchitis, and other diseases and conditions. In its method aspects, the present invention includes inhaling (or forcing manually or automatically) cool and/or moist air from the device to cause cool and/or moist air to enter the airways of the patient.

### BACKGROUND OF THE INVENTION

The present invention relates to inhalation apparatus for the relief from symptoms of various respiratory illnesses, and to methods of treating such symptoms.

There are a number of diseases and conditions that may cause upper respiratory tract symptoms that result in considerable discomfort. Exemplary diseases and conditions include the common cold, croup, laryngitis, Laryngotracheobronchitis, bronchitis, strep throat, mononucleosis, whooping cough (pertussis), respiratory tract infections, respiratory syncytial virus (RSV), flu, pneumonia, allergies, asthma, tonsillitis, etc. Exposure to severe environmental conditions, such as excessive heat during a fire or exposure to toxic gases may result in similar symptoms and cause similar discomfort.

Various remedies that attempt to reduce or alleviate such discomfort are available, including vapor rubs, cough drops and lozenges, humidifiers, vaporizers, etc. These remedies, however, suffer from various disadvantages. For example, humidifiers, cool mist vaporizers or ultrasonic nebulizers require confining the patient to a relatively small area, as does the use of a hot shower, which confines the patient to a space that is relatively small and that can be very uncomfortable. Bacteria and fungi can grow in the filters and water tanks of portable and console humidifiers, and can be aerosolized and distributed throughout the room. Cough drops and lozenges can have high sugar content and can be high in calories.

It would therefore be desirable to provide a device that is inexpensive and convenient to use, and that reduces or eliminates the foregoing discomforts without suffering from the drawbacks of the prior art.

### SUMMARY OF THE INVENTION

The problems of the prior art have been overcome by the present invention, which provides an inhaler device and a method of treating various symptoms associated with the airway and/or throat and/or respiratory system of a patient. The device is particularly designed to reduce swelling and inflammation of the larynx and or upper respiratory track, such as swelling and/or inflammation that result from croup, laryngitis's, laryngotracheobronchitis, and other diseases and conditions including those mentioned above. The device does not require that the user wear a mask.

In certain embodiments, the device includes a housing containing a low temperature material and having an air flow passageway, such that when a patient inhales from the device, air travels into the air flow passageway and is cooled (and/or humidified) by the low temperature material as it travels through the device. The now cooled air exits the device and enters the airways of the patient.

In its method aspects, the present invention includes inhaling (or forcing manually or automatically) cool and/or moist air from the device to cause cool and/or moist air to enter the airways of the patient.

The device can be hand-held, or can be supported on a substrate such as the floor, a table or similar stand.

### BRIEF DESCRIPTION OF THE INVENTION

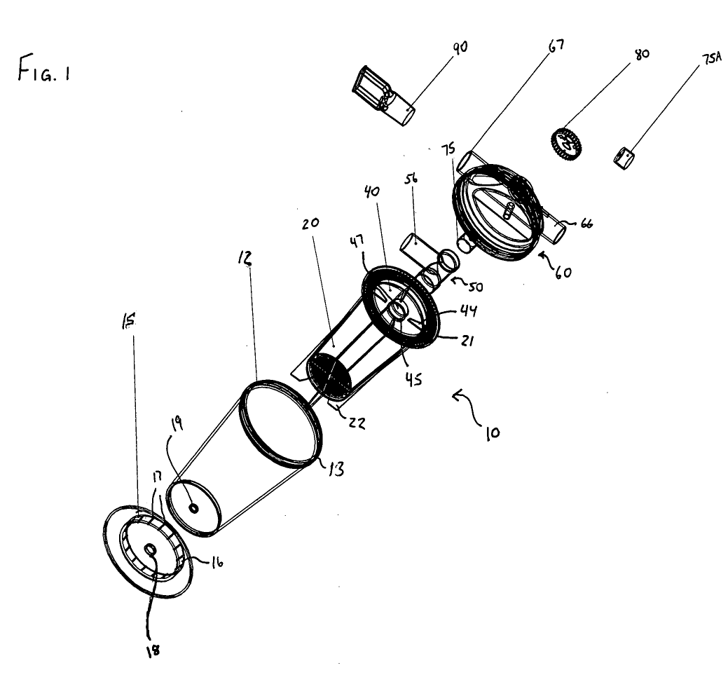

FIG. 1 is an exploded view of a device in accordance with certain embodiments;

FIG. 2 is a cross-sectional view of the device in its assembled condition in accordance with certain embodiments;

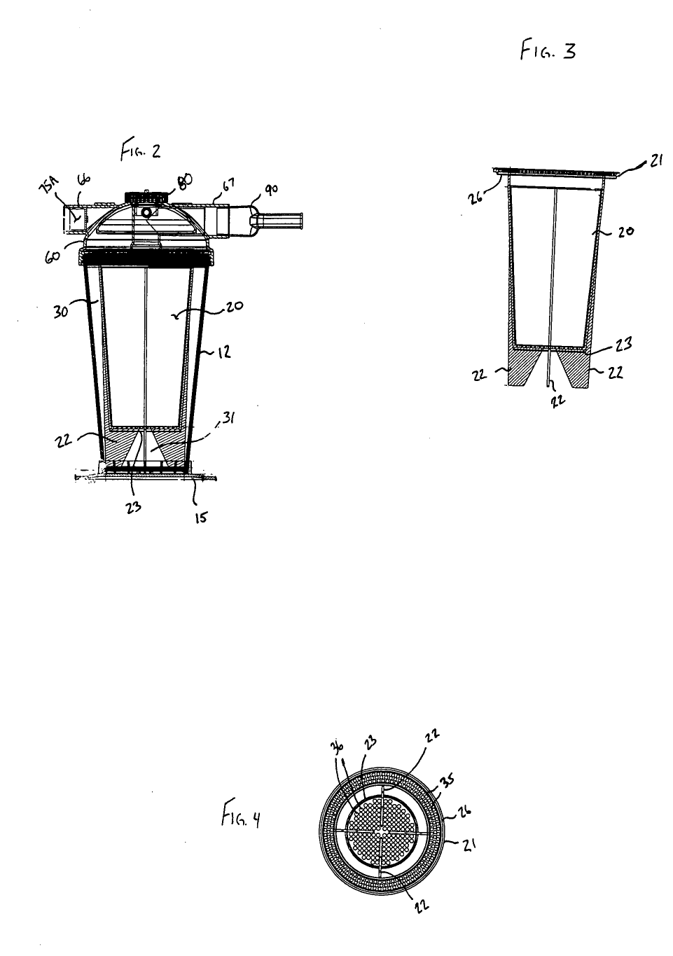

FIG. 3 is a cross-sectional view of an inner housing of the device in accordance with certain embodiments;

FIG. 4 is a bottom view of the inner container of FIG. 3;

FIG. 5 is a cross-sectional view of an insert for the inner housing in accordance with certain embodiments;

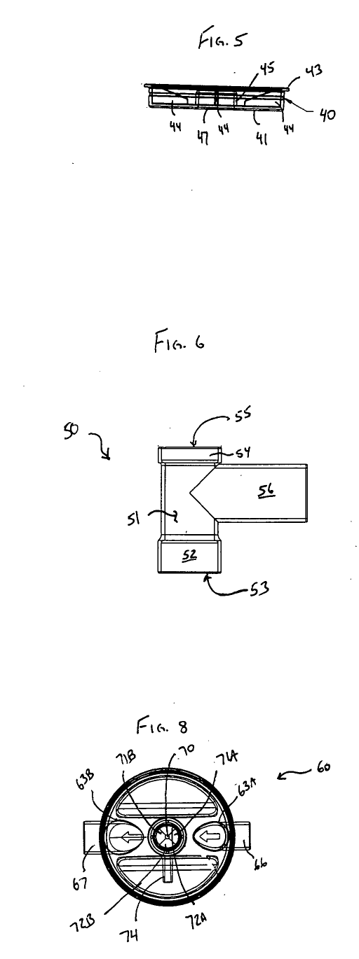

FIG. 6 is a side view of a center pipe in accordance with certain embodiments;

FIG. 7 is a cross-sectional view of the cover of the device in accordance with certain embodiments;

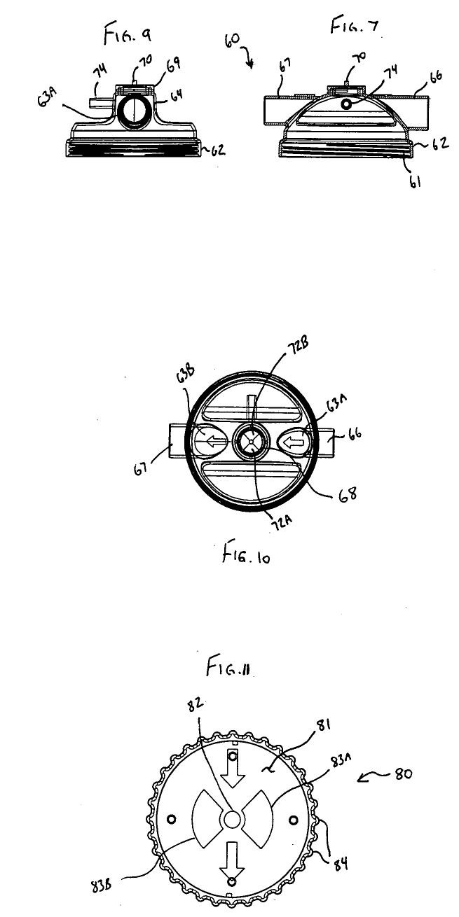

FIG. 8 is a top view of the cover of FIG. 7;

FIG. 9 is a side view of the cover of FIG. 7;

FIG. 10 is a bottom view of the cover of FIG. 7;

FIG. 11 is a top view of the twist cap in accordance with certain embodiments;

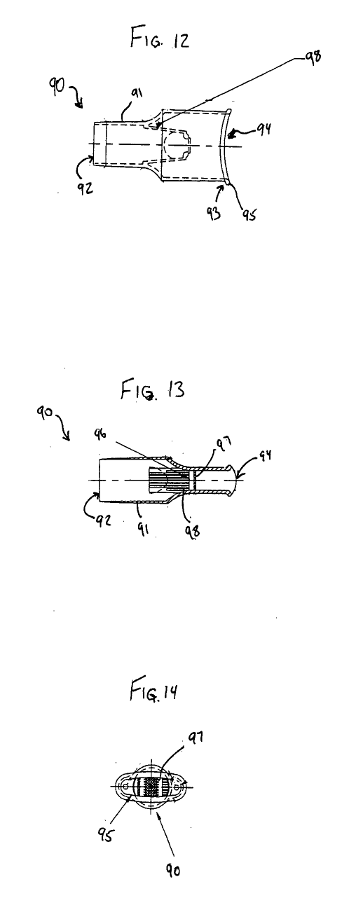

FIG. 12 is a cross-sectional top view of a mouthpiece in accordance with certain embodiments;

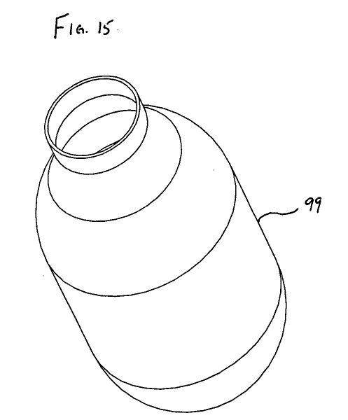

FIG. 13 is a cross-sectional side view of a mouthpiece in accordance with certain embodiments;

FIG. 14 is a front view of the mouthpiece in accordance with certain embodiments; and

FIG. 15 is a perspective view of an air bulb suitable for use with certain embodiments.

### DETAILED DESCRIPTION OF THE INVENTION

Turning first to FIG. 1, there is shown a device 10 in accordance with certain embodiments of the present invention. The device 10 includes an outer housing 12 that is shown in the shape of a truncated cone, tapering from the top end towards the bottom end. Those skilled in the art will appreciate that other shapes, including cylinders, rectangles, and irregular polygons are suitable and are within the scope of the invention. Preferably the outer housing is rigid, liquid impervious, and is durable. Suitable materials include plastics, polypropylene, polyvinyl chloride, polytetrafluoroethylene (TEFLON), polyethylene, polycarbonate, styrofoam, polystyrene, acrylic, metal, glass, etc. The most preferred material is FDA approved low density polyethylene. The outer housing 12 can be made to have antimicrobial and/or antibacterial properties, such as by coating or embedding an antimicrobial and/or antibacterial agent therein. The outer housing 12 is open at its top end and closed at its bottom end, and can include external threads 13 at or near the top end for mating with a cover as described in greater detail below.

A disc-shaped removable base 15 may be provided to help support and balance the assembly in an upright position. The base 15 is preferably circular, having a diameter larger than the diameter of the base of the outer housing 12. A raised annular collar 16 having a diameter slightly larger than the diameter of the bottom of the outer housing can be provided on the base 15 and is used to receive or engage the outer housing 12 to support the same in the base 15. A plurality of inner ribs 17 help secure the outer housing 12 in the annular collar 16. To further secure the outer housing 12 in the base 15, a second smaller raised annular collar 18 can be provided in the base 15 to mate with a smaller raised annular collar 19 formed in the bottom of the outer housing 12. The outer diameter of the collar 19 is slightly smaller than the inner diameter of the second raised annular collar 18 so that it fits snuggly within the annular collar 18 by frictional engagement. Those skilled in the art will appreciate that the foregoing combination of annular collars are merely exemplary ways to secure the outer housing 12 to the base 15, and that alternative mechanisms can be used and are within the scope of the invention.

An air flow passageway is formed in the outer housing 12 such as with an inner housing 20 defining a perforated chamber, as best see in FIGS. 1-3. Preferably the inner housing 20 has a general shape substantially similar to that of the outer housing 10, and most preferably it is shaped as a truncated cone, tapering from the top end towards the bottom end. Preferably the inner housing is rigid, liquid impervious, is durable, and can be made of the same materials suitable for the outer housing 12. An FDA approved low density polyethylene is particular preferred. It also can be modified to have antimicrobial and/or antibacterial properties. The inner housing 20 is generally smaller in diameter than the outer housing 12, such that the outer housing can contain the inner housing when the device 10 is assembled, and such that a gap 30 between the wall of the outer housing 12 and the wall of the inner housing 20 is formed defining the air flow passageway. The inner housing 20 includes a top annular collar or flange 21 that has a greater diameter than the remainder of the housing 20. Preferably the diameter of the collar 21 is about equal to the diameter of the top rim of the outer housing 12, such that when the inner housing 20 is placed in the outer housing 12, the collar 21 sits on the top rim of the outer housing 12 and is supported thereby. The bottom of the collar 21 can include a radially inward annular ring 26 that is spaced from the circumferential outer end of the collar a sufficient distance such that when the inner housing 20 is positioned in the outer housing 12, the annular ring 26 abuts against the inner wall of the outer housing 12, just below the top rim thereof. Alternatively or in addition, the inner housing 20 can include an external support, such as one or more legs 22, which extend below the base 23 of the inner housing 20 and are supported on the base of the outer housing 12. In certain embodiments, four such legs 22 are provided, symmetrically about the inner housing 20. The legs 23 can be integrally molded to the outside of the inner housing 20, or can be attached by any suitable means, such as with an adhesive.

Preferably the perforations or apertures 36 in the inner housing 20 are provided in the base 23 thereof, so that air entering the inner housing 20 through the apertures has the optimum residence time in the inner container 12 for contact with the low temperature material. Indeed, locating the apertures at the base 23 of the inner housing 20 also ensures that as the air travels down the air flow passageway defined by gap 30, it contacts the outer surface of the inner housing. Since the outer surface of the inner housing may also be cooled by the low temperature material, the cooling of the air begins as the air flows downwardly to the base of the inner container, even before it enters the inner container and contacts the low temperature material. However, it is within the scope of the invention to provide the perforations elsewhere on the inner container, such as along the side wall of the inner container 20 instead of, or in addition to, perforations in the base 23 thereof. It is also within the scope of the invention to construct the inner housing 20 out of a thermally conductive material, such as stainless steel, so that the air traveling in the air flow passageway defined by gap 30 is even more effectively cooled by the low temperature material contained in the inner housing.

It is important for proper operation of this embodiment of the device that the inner housing 20 be positioned in the outer housing 12 such that a sufficient volume 31 is provided between the base 23 of the inner housing 20 and the base of the outer housing 12, to allow air to flow from the gap 30 to the volume 31 and then into the inner housing 20 through apertures 36 in the base 23, and providing a melt chamber for melted ice to collect while still ensuring air flow into the inner housing through the apertures 36. For example, the legs 23, when provided, should extend below the base 23 of the inner housing a sufficient amount, and that the height of the inner housing be such that, a sufficient volume 31 is provided between the base 23 of the inner housing 20 and the base of the outer housing 12 as seen in FIG. 2. The interior volume of the inner housing 20 is configured to hold a low temperature material or coolant (i.e., a material having a lower temperature than the ambient air, preferably at least 10-20° lower, more preferably at least about 30° lower), such as ice, cold packs (e.g., ammonium nitrate based packs), gel packs, etc., preferably ice. The ice may be in the form of cubes, crushed ice or a single block of ice, and is preferably supported on the base 23 of the inner housing 20. The volume 31 should be sufficient to hold any water that results from the melting of the low temperature material, while still maintaining sufficient space to allow air flow into the inner container without causing the water to be carried along with the air flow, which can cause the user to gurgle.

Although the inner housing 20 is preferably removable from the outer housing 12, it is within the scope of the invention to provide a single or integral housing that includes interior walls that define the gap 30 and volume 31 and thus performs the same function as two separate housings 12 and 20 as described above.

Turning now to FIG. 4, the collar 21 is shown having a plurality of spaced apertures 35 positioned annularly around the collar 21. In the embodiment shown, there are three concentric rings of circular apertures 35, with a first outer ring radially inwardly of the ring 26 near the outer circumferential end of the collar 21, a second middle ring radially inwardly of the first outer ring, and a third inner ring radially inwardly of the second middle ring. Preferably the apertures 35 in each ring are substantially evenly spaced, and have diameters of about 2.5 mm. Preferably each ring of apertures is also substantially evenly spaced from an adjacent ring. Those skilled in the art will appreciate that additional or fewer apertures and/or rings of apertures can be provided without departing from the spirit and scope of the invention, and that the shape of the apertures is not particularly limited. It also will be readily appreciated that the invention is not to be limited to any particular pattern of apertures; other patterns or randomly oriented apertures also may be used, keeping in mind that it is important that a sufficient number and size of the apertures should be provided to allow for sufficient air flow through the apertures as discussed in greater detail below.

Since the collar 21 extends radially outwardly from the interior of the inner container 20, when the inner container 20 is properly positioned in the outer container 12, the collar, via apertures 35, allows air flow into the gap 30 from either intake opening aperture 63A in cover 60, or port 74 in cover 60 (discussed in greater detail below).

FIG. 4 also shows that the base 23 of the inner housing 20 includes a plurality of spaced apertures 36. The number and size of the apertures 36 also should be sufficient to ensure sufficient air flow through the apertures as discussed in greater detail below. Preferably the apertures 36 are circular and are located substantially over the entire base 23 surface, and are 2.88 mm in diameter. Other suitably shaped apertures can be used. The numbers and sizes of the apertures 35 and apertures 36 are preferably chosen so that the air flow through the apertures 35 is substantially the same as the air flow through the apertures 36.

FIG. 5 shows a center insert 40 for the inner housing 20. The insert 40 is preferably made of the same materials used to make the outer housing 12. It includes a base 41 and an annular side wall 42 that extends upwardly from the base 41 and terminates in a circumferential top flange 43 that extends radially outwardly as shown. The outer diameter of the annular side wall 42 is slightly smaller than the inner diameter at or near the top of the inner container 20, such that the insert 40 fits snuggly inside the inner container 12 by frictional engagement when in the assembled condition. It is readily removable from the container 20 so as to provide access to the inside of the container 20 such as to position low temperature material (e.g., ice) in the container 20. The diameter of the circumferential top flange 43 is greater than the inner diameter at or near the top of the inner container 20, such that the flange 43 acts as a stop, seating on the top surface of the collar 21 (without blocking any of the apertures 35) and preventing the insert from descending further into the inner housing 20. The insert 40 may include one or more ribs 44 (e.g., four symmetrically spaced) to add structural integrity thereto. The insert 40 also includes an aperture 47, preferably centrally located in the insert, and an annular upwardly extending wall 45 circumscribing the aperture 47. The insert 40 includes a blocking portion that serves to block air flow from entering the interior of the inner container 20 other than through aperture 47 (i.e., prevents air from entering the interior of the inner container 20 without first passing through the air passageway defined by the gap 30).

The annular wall 45 of insert 40 is configured to receive center pipe 50 (FIG. 6) in frictional engagement. Center pipe 50 includes a central pipe 51, including a base portion 52 having an open end at 53 and a top portion 54 having an open end at 55. The base portion 52 mates with the annular wall 45 of insert 40, such as by configuring the inner diameter of the base portion 52 to be slightly greater than the outer diameter of the wall 45, so that the base portion 52 can receive the wall 45 and allow fluid communication between the inner housing 20 and the center pipe 51 via aperture 47 in the insert 40. Extending radially from the center pipe 50 is radial arm 56, which can be integrally molded to the center pipe or affixed thereto such as with an adhesive. The radial arm 48 is configured to be received in conduit 67, providing fluid communication between the outtake of the device and the inner container 20 as discussed in greater detail below.

FIGS. 7-10 illustrate the removable cover 60 of the device 10. Preferably the cover 60 includes an annular side wall 62 having internal threads 61 for cooperating with external threads 13 on the outer housing 12 such that the cover can be secured, by screwing, to the outer housing 12. Other means of attachment of the cover 60 to the outer housing 12, such as by snapping, can be used. As best seen in FIG. 9, preferably the cover 60 includes a central raised portion 64, that as two spaced side apertures 63A, 63B FIG. 8) and central apertures 72A, 72B. Conduit member 66 is secured in intake aperture 63A, such as by gluing, or can be integrally formed as part of the cover. Similarly, conduit member 67 is secured in outtake aperture 63B. An optional port 74 may be provided in the cover 60, and in the embodiment shown, extends radially outwardly from the central raised portion 64. A cap (not shown) or the like can be provided to close off the open end of the port 74 depending upon the mode of use.

As seen in FIGS. 8 and 9, extending upwardly from the central raised portion 64 is a disk member 69, having a central upwardly extending pin 70. The disk 69 includes a pair of oppositely spaced pie shaped solid members 71A, 71B that co-join at the location of central pin 70. The oppositely spaced pie shaped solid members 71A and 71B define between them the oppositely spaced pie shaped central apertures 72A, 72B that together define an exhaust port. Other configurations of the exhaust port can be used.

As best seen in FIG. 10 viewing the cover 60 from the underside, central aperture 63C is defined by a ring 68 that has an annular side wall that extends downwardly from the top of the disk 69. The ring 68 is dimensioned to be received by the top portion 54 of the central pipe 50. Thus, the ring 68 has an external diameter slightly smaller than the internal diameter of the top portion 54 of the central pipe 50 for frictional engagement therewith.

Ring 68 (and thus top portion 54) is also dimensioned to receive a one-way valve 75. One suitable one-way valve is an AirLife™ one-way valve commercially available from Cardinal Health Respiratory Care. The one-way valve includes a cylindrical support, supporting a donut-shaped membrane that allows air flow through it only one direction. The valve is snuggly disposed in ring 68 by frictional engagement such that the membrane is positioned just beneath the apertures 72A, 72B, allowing air flow through it and out the apertures 72A, 72B. A. similar one-way valve 75A (FIG. 1) is positioned in conduit member 66 to allow air flow into the device 10 and thus provides an air intake.

FIG. 11 illustrates twist cap 80 that is rotatably disposed on disk member 69, forming a valve assembly to open and close the exhaust port. The top surface 81 of the cap 80 includes a central aperture 82 for receiving central pin 70 to properly position the cap 80 on the disk member 69 and help secure it thereto. The top surface 81 also includes a pair of spaced arc-shaped apertures 83A, 83B, each positioned to respectively align with one of apertures 72A, 72B when the cap 80 is in a first position with respect to the disk member 69 (thereby allowing air flow (e.g., air exhaled by the user) out of the exhaust port), and to respectively align with one of pie-shaped members 71A, 71B when the cap 80 is in a second position with respect to disk member 69 (thereby preventing air flow out of the exhaust port). The cap 80 may include one or more knurls 84 formed on the outer perimeter thereof to facilitate manual rotation of the cap 80 about disk member 69. Those skilled in the art will appreciate that other valve assemblies can be used to control the opening and closing the exhaust port. The one-way valve 75 allows air to travel only out of the exhaust port; air cannot travel into the device through the exhaust port.

Turning now to FIGS. 12 and 13, a mouthpiece 90 suitable for use with the device of the invention is shown. Preferably the mouthpiece 90 is made out of a polyolefin, most preferably polypropylene. A suitable mouthpiece is commercially available from Teleflex Incorporated. The mouthpiece 90 has a cylindrical portion 91 having an open end 92. The outside diameter of the cylindrical portion 91 is preferably slightly smaller than the inside diameter of conduit 67, so that mouthpiece 90 can be inserted into conduit 67 and fits snuggly within conduit 67 and remains in the conduit by frictional engagement unless it is forceably (such as by manually pulling on the mouthpiece) removed therefrom. The mouthpiece is removable from the conduit 67 and can be disposed of, and then replaced by a new mouthpiece so that a different user may use the device without risking contamination from other users. It is noted that the inside diameter of conduit 67 is preferably different from the inside diameter of conduit 66, so that the mouthpiece fits in only conduit 67 and cannot mistakenly be placed in conduit 66. In the embodiment shown, the inside diameter of the conduit 67 is larger than that of conduit 66.

The distal end 93 of the mouthpiece 90 has an oval shaped opening 94 shaped to the average general contour of a person's mouth (FIG. 14), defined by an outwardly extending flange portion 95 adapted to fit into the patient's mouth and around which the lips of the patient can be positioned so comfortably position and maintain the mouthpiece in the patient's mouth.

Mouthpiece 90 preferably has an internal filter assembly 96, integrally molded therein. In the embodiment shown, the filter assembly 96 includes a front checkerboard grate 97, and opposite side filters 98 tapering outwardly as they extend towards the end 92 of the mouthpiece 90. The filter assembly 96 helps ensure that no particular matter enters the patient's mouth during use of the device, and is shaped to direct the air flow towards the center of the mouthpiece. Those skilled in the art will appreciate that other filter assemblies could be used without departing from the spirit and scope of the invention.

In an alternative embodiment, the mouthpiece 90 can be attached to suitable flex medical tubing (not shown), which tubing is then attached to the device via conduit 67. This allows the device 10 to be positioned further away from the patient's mouth if desired. In yet a further alternative embodiment, a facemask can be placed in fluid communication with the device and can be worn by the user to cover the user's mouth, nose or both. Alternatively still, a nasal cannula can be positioned in fluid communication with the conduit 67 to direct air into the nose of the patient.

The device can be assembled by inserting the one-way valves in the appropriate locations, and attaching the center pipe 50 to the cover by inserting radial arm 56 in conduit 67 and top portion 54 about ring 68. Inner housing 20 is placed inside outer housing 12, and cover 60 is attached to the outer housing. Attachment of the cover 60 to the outer housing 12 causes the base portion 52 of the center pipe 50 to engage the annular upwardly extending wall 45 of insert 40, thereby establishing fluid communication between the interior of the inner housing 20 and the center pipe 50.

In use in a first mode of operation, a low temperature material such as ice is placed in the inner container 20, and the device is assembled. The amount of low temperature material is not particularly limited, although it is preferred that when ice is used, the container 20 be filled in order to maximize the length of time the ice remains solid. The cap 80 is positioned so that the exhaust port is open, allowing one-way flow out the exhaust port through one-way valve 75. The air port 74 is closed. The patient then places his mouth about the mouthpiece 90, and inhales. The inhalation is a driving force that draws ambient air into the device 10 through the intake opening in conduit 66, through one-way valve 75A positioned therein, down through apertures 35 and through the gap 30 between the inner housing 20 and the outer housing 12, into the volume 31, up through apertures 36 in the base 23 of the inner housing 20 and into the inner housing 20, where the air contacts the low temperature material which effectively lowers the temperature of and adds humidity to the air. The now cool, moist air exits the inner housing 20, flows into central pipe 51 of center pipe 50, into radial arm 56 thereof, and then through the outtake opening 63B and into the mouthpiece 90, from which it enters the mouth of the patient, where it cools the throat, larynx, upper respiratory tract, etc. of the patient, providing soothing relief thereto. The intake opening and outtake opening, although in fluid communication via the gap 30 and the inner housing 20, are segregated; intake air must flow through the inner housing 20 (and thus be cooled by the low temperature material) prior to reaching the outtake. The patient may then exhale into the mouthpiece, and the flow of exhaled air travels through conduit 67, up through center pipe 50, through one-way valve 75, and then through the exhaust port to ambient (the one-way valve 75A prevents exhaled air from exiting through the conduit 66. In this mode, the patient does not have to remove his mouth from the mouthpiece 90 to exhale; the patient can breath (inhale and exhale) normally while maintaining the mouthpiece inside the mouth. A closed system is thus established. Alternatively, the patient may remove the mouthpiece from the mouth and exhale into the ambient environment rather than into the device 10. As the ice melts, the resulting liquid drips into and collects in the volume 31 between the inner container 20 and outer container 12, which can be periodically emptied. The inner container 20 can be periodically replenished with ice and/or other low temperature material as needed simply by removing the cover 60 and insert 40.

In an alternative mode of operation, particularly applicable for use with small children (e.g., under 3 years of age), infants or the elderly, an air assist can be provided. In this mode, the exhaust port is closed by moving disk member 80 so that its pie-shaped apertures 83A, 83B are out of alignment with apertures 72A, 72B, thereby blocking flow through apertures 72A, 72B. The air port 74 is also closed. A driving force, such as a manually operated air bulb 99 (FIG. 15), pump or fan, is then placed in fluid communication with the conduit 66 (such as through suitable tubing (not shown)), and is actuated such that air is forced into the conduit 66 and through one-way valve 75A in the intake aperture 63A. The air then flows down through apertures 35 and through the air passageway defined by gap 30 between the inner housing 20 and the outer housing 12, up through apertures 36 in the base 23 of the inner housing 20 and into the inner housing 20, where the air contacts the low temperature material which effectively lowers the temperature of and adds humidity to the air. The now cool, moist air exits the inner housing 20, flowing into central pipe 51 of center pipe 50, into radial arm 56 thereof, and then through the mouthpiece 90 and into the mouth of the patient. It should be noted that the mouthpiece need not be positioned inside the mouth of the patient; it is sufficient to place the mouthpiece (or, for example, the outlet of conduit 67) in proximity to the mouth and/or nose of the patient (e.g., within about 3 inches thereof) to effectively cool the air that the patient is breathing. Indeed, this allows the patient to breath normally without forcing too much cool air into the respiratory system of the patient. It should be noted that the mouthpiece can be omitted entirely in this mode. Alternatively still, a nosepiece such as a nasal cannula (not shown) can be used and can be inserted into the nose of the patient or placed in proximity thereto. The air assist as the driving force for air circulation through the device can be provided alone or in addition to inhalation by the patient.

In yet a further alternative mode of operation, the exhaust port is closed by moving disk member 80 so that its pie-shaped apertures 83A, 83B are out of alignment with apertures 72A, 72B, thereby blocking flow through apertures 72A, 72B. The air port 74 is opened, and an air or oxygen feed source, such as a typical oxygen feed available in hospitals, is placed in fluid communication with the port 74. In the hospital, oxygen conventionally is supplied to each patient room and is available via an outlet in the wall, and a flow meter and valve regulate the oxygen flow. Attachments may be connected to moisturize the oxygen flow. In the home, the oxygen source is usually an oxygen canister or an air compressor. This mode allows for a constant flow of oxygen to enter the device and be cooled by flowing through the device as discussed above.

The inhaler could also be used to warm air by substituting a high temperature material, such as a pouch containing iron powder that when exposed to air, causes an oxidation reaction producing heat, for the low temperature material.

1. Inhaler apparatus, comprising:
an outer housing;
an inner housing adapted to be positioned in said outer housing such that said inner and outer housings define therebetween an air flow passageway; said inner housing comprising a perforated chamber adapted to contain a low temperature material; and
a cover having an intake opening adapted to be in fluid communication with said air flow passageway, and an outtake opening in fluid communication with said perforated chamber.

2. The inhaler apparatus of claim 1, further comprising an exhaust port.

3. The inhaler apparatus of claim 1, whereby air drawn into said apparatus via said intake opening flows into said air flow passageway, into said inner housing via the perforations in said perforated chamber where it is cooled by said low temperature material, and out said inner housing and through said outtake opening.

4. The inhaler of claim 1, wherein said inner housing comprises a collar having at least one aperture allowing fluid communication between said intake opening and said air flow passageway.

5. The inhaler of claim 4, wherein said inner housing further comprises an insert having an aperture and a blocking portion, wherein said blocking portion prevents air that enters said intake opening from entering said inner housing prior to passing through said air flow passageway.

6. The inhaler of claim 1, further comprising a driving force for forcing air into said intake opening.

7. The inhaler of claim 6, wherein said driving force comprises an air bulb.

8. The inhaler of claim 2, further comprising a valve for selectively opening and closing said exhaust port.

9. A method of cooling the airways of an individual, comprising:
providing an inhaler comprising a chamber for a low temperature material, an intake opening, an outtake opening, an exhaust port, and an air passageway segregating said intake opening from said outtake opening and providing fluid communication between said intake opening and said outtake opening through said chamber;
inhaling ambient air through said intake opening, causing said inhaled air to flow through said air passageway and into said chamber, be cooled by said low temperature material contained in said chamber, and flow out of said chamber into said outtake opening; and
exhaling air into said outtake opening, causing said exhaled air to flow through said outtake opening and out said exhaust port.

10. An inhaler, comprising:
a housing having a chamber adapted to contain a low temperature material; said chamber being in fluid communication with ambient air via a one-way valve, said ambient air upon activation of a driving force being drawn into said chamber and being cooled by said low temperature material, said cooled air then flowing out of said chamber.

11. The inhaler of claim 10, wherein said driving force is created by the inhalation by a user of said inhaler.

12. The inhaler of claim 10, wherein said driving force comprises an air bulb.

13. The inhaler of claim 10, wherein said housing further comprises an exhaust port for exhausting exhaled air from said inhaler.

14. The inhaler of claim 13, further comprising a valve for selectively opening and closing said exhaust port.

15. The inhaler of claim 10, wherein said chamber is perforated.

16. The inhaler of claim 15, whereby air drawn into said housing via said one-way valve flows into said chamber via said perforations in said chamber where it is cooled by said low temperature material, and flows out said chamber in a cooled state.

17. Inhaler apparatus, comprising an air intake, an air outtake, a perforated chamber adapted to contain a low temperature material, a melt chamber, and an air flow passageway providing fluid communication between said intake and said perforated chamber, said melt chamber being positioned to collect melting fluid from said low temperature material.

18. Inhaler apparatus of claim 17, wherein said perforated chamber is in fluid communication with said air intake via a one-way valve.

19. Inhaler apparatus of claim 17, further comprising an exhaust port.

20. Inhaler apparatus of claim 19, further comprising a valve for selectively opening and closing said exhaust port.

21. A method of providing humidified air to the airways of an individual, comprising:
providing an inhaler comprising a chamber for a low temperature material, an intake opening, an outtake opening, and an air passageway segregating said intake opening from said outtake opening and providing fluid communication between said intake opening and said outtake opening through said chamber; and
inhaling ambient air through said intake opening, causing air to flow through said air passageway and into said chamber, be humidified by said low temperature material contained in said chamber, and flow out of said chamber into said outtake opening.

22. The method of claim 21, wherein said inhaler further comprises an exhaust port, and wherein after said inhaling step, further comprising the step of exhaling air into said outtake opening, causing said exhaled air to flow through said outtake opening and out said exhaust port.

23. The method of claim 21, wherein said low temperature material is ice.

24. The method of claim 21, wherein said individual inhales through a facemask in fluid communication with said intake opening.

25. The inhaler apparatus of claim 1, further comprising a facemask in fluid communication with said intake opening.
